(19) United States
(12) Patent Application Publication
(10) Pub. No.: US 2006/0183106 A1
(43) Pub. Date: Aug. 17, 2006

(54) Processes for identifying quadruplex-targeted antiviral molecules

(76) Inventor: Adam Siddiqui-Jain, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 10/531,545

(22) PCT Filed: Oct. 20, 2003
(86) PCT No.: PCT/US03/33366

§ 371 Date: Apr. 15, 2005

Related U.S. Application Data
(60) Provisional application No. 60/419,456, filed on Oct. 18, 2002.

(51) Int. Cl. C12Q 1/70; C12Q 1/68

(52) U.S. Cl. 435/5; 435/6

(57) ABSTRACT

Featured herein are processes useful for identifying candidate molecules that interact with a quadruplex structure formed in the central flap nucleic acid of retroviruses. The candidate molecules identified by the process may be used as antiviral agents.

### FIELD OF THE INVENTION

The invention concerns methods for identifying molecules that interact with a viral nucleic acid capable of forming a G-quadruplex secondary structure.

### BACKGROUND

Developments in molecular biology have led to an understanding of how certain therapeutic compounds interact with molecular targets and lead to a modified physiological condition. Specificity of therapeutic compounds for their targets is derived in part from interactions between complementary structural elements in the target molecule and the therapeutic compound. A greater variety of target structural elements in the target leads to the possibility of unique and specific target/compound interactions. Because polypeptides are structurally diverse, researchers have focused on this class of targets for the design of specific therapeutic molecules.

In addition to therapeutic compounds that target polypeptides, researchers also have identified compounds that target DNA. Some of these compounds are effective anticancer agents and have led to significant increases in the survival of cancer patients. Unfortunately, however, these DNA targeting compounds do not act specifically on cancer cells and therefore are extremely toxic. Their unspecific action may be due to the fact that DNA often requires the uniformity of Watson-Crick duplex structures for compactly storing information within the human genome. This uniformity of DNA structure does not offer a structurally diverse population of DNA molecules that can be specifically targeted.

Nevertheless, there are some exceptions to this structural uniformity, as certain DNA sequences can form unique secondary structures. For example, intermittent runs of guanines can form G-quadruplex structures, and complementary runs of cytosines can form i-motif structures. Formation of G-quadruplex and i-motif structures occurs when a particular region of duplex DNA transitions from Watson-Crick base pairing to intermolecular and intramolecular single-stranded structures.

### SUMMARY

Certain regulatory regions in retroviral nucleic acids can adopt single-stranded G-quadruplex structures. For example, a single-stranded tail incorporated within the center of the double-stranded transcription product of a retroviral nucleic acid, designated the central flap region, is responsible for multimerization of the retroviral nucleic acid. The central flap is capable of adopting a quadruplex structure that can interact with the quadruplex of another central flap thereby facilitating multimerization of the retroviral nucleic acid. Destabilizing the multimer into individual viral nucleic acids is an event necessary for viral replication in host cells. Accordingly, molecules that interact with and stabilize the central flap quadruplexes can act as antiviral therapeutics when administered to a system or animal infected with a retrovirus.

Thus, featured herein is a method for identifying an antiviral candidate molecule, which comprises contacting a test molecule with a nucleic acid comprising a nucleotide sequence identical to or substantially identical to a nucleotide sequence in a retroviral central flap nucleic acid capable of forming a quadruplex structure, and detecting an interaction between the test molecule and the nucleic acid. A test molecule that interacts with the nucleic acid is considered an antiviral candidate molecule. The nucleic acid sometimes forms an intermolecular quadruplex structure, which sometimes is a parallel intermolecular structure. The nucleic acid sometimes forms an intramolecular quadruplex structure, which sometimes is an intramolecular antiparallel structure (e.g., a chair conformation having bridging loops running orthogonal to two parallel loops and resulting from the simple folding-over of a DNA G-hairpin). Where the nucleic acid adopts an intramolecular antiparallel structure, intermolecular dimeric structures sometimes are formed.

In an embodiment, the nucleotide sequence is from human immunodeficiency virus (HIV, e.g., HIV-1 or HIV-2) and comprises the sequences TTG₆TA, CAG₄AA, or both. In the latter nucleotide sequences, one or more nucleotides flanking the G₆ and G₄ stretches sometimes are substituted by another nucleotide (e.g., one or more of the TT or TA flanking the G₆ stretch or CA or AA flanking the G₄ stretch sometimes are substituted by another nucleotide). In related embodiments, the nucleic acid includes additional sequences flanking the G₆ or G₄ stretches from an HIV strain, and in specific embodiments, the nucleic acid comprises or consists of the nucleotide sequence TTGGGGGGTACAGTGCAGGGGAA.

The interaction sometimes is detected by monitoring polymerase arrest as a result of quadruplex stabilization induced by the test molecule, and often the interaction is detected by circular dichroism. In circular dichroism (CD) embodiments, changes in CD often are monitored as a function of temperature. Processes for identifying candidate molecules often are performed in vitro (e.g., in a cell-free environment or in cells) and sometimes are performed in vivo (e.g., in a tissue, organ or a subject such as a mouse, rat, rabbit, hamster, monkey or human).

Also featured are methods for treating a retroviral infection by administering a candidate molecule identified by the processes described herein to a subject in need thereof. Such methods can provide a substantial advantage over other anti-viral therapeutics. For example, current Highly Active Anti-Retroviral Therapeutic (HAART) regimes rely on the use of combinations of drugs targeted towards the HIV protease and HIV integrase. The requirement for multi-drug regimes is to minimize the emergence of resistance, which will usually develop rapidly when agents are used in isolation. The source of such rapid resistance is the infidelity of the reverse transcriptase enzyme which makes a mutation approx. once in every 10,000 base pairs. An advantage of targeting critical viral quadruplex structures over protein targets, is that the development of resistance is slow or is impossible. A point mutation of the target quadruplex, necessary to reduce affinity for the candidate molecule, can compromise the integrity of the critical quadruplex structure and lead to a non-functional copy of the virus. A single therapeutic agent based on the embodiments described herein can replace the multiple drug regimes currently employed, with the concomitant benefits of reduced costs and the elimination of harmful drug/drug interactions.

### DETAILED DESCRIPTION

Featured herein is a screening process useful for identifying candidate antiviral molecules that interact with quadruplex-forming nucleic acids comprising a nucleotide sequence identical to or substantially identical to a nucleotide sequence from a retroviral central flap nucleic acid. Some or these molecules are expected to be useful as antiviral therapeutics, such as therapeutics for treating a retroviral infection.

Nucleic Acids

The first nucleic acid and second nucleic acid are independently selected from the nucleic acids described below. Nucleic acids often comprise or consist of DNA (e.g., genomic DNA (gDNA) or complementary DNA (cDNA)) or RNA (e.g., mRNA, tRNA, and rRNA). In embodiments where a nucleic acid is a gDNA or cDNA fragment, the fragment often is 50 or fewer, 100 or fewer, or 200 or fewer base pairs in length, and sometimes is about 300, about 400, about 500, about 600, about 700, about 800, about 900, about 1000, about 1100, about 1200, about 1300, or about 1400 base pairs in length. In an embodiment, the nucleic acid is double-stranded, and is sometimes between about 30 nucleotides to about 40 nucleotides in length. Methods for generating gDNA and cDNA fragments are known in the art (e.g., gDNA may be fragmented by shearing methods and cDNA fragment libraries are commercially available). In embodiments where the nucleic acid is a synthetically prepared fragment nucleic acid, often referred to as an “oligonucleotide,” the fragment sometimes is less than 30, less than 40, less than 50, less than 60, less than 70, less than 80, less than 90, or less than 100 nucleotides in length. Synthetic oligonucleotides can be synthesized using standard methods and equipment, such as by using an ABI™ 3900 High Throughput DNA Synthesizer, which is available from Applied Biosystems (Foster City, Calif.).

Nucleic acids sometimes comprise or consist of analog or derivative nucleic acids, such as peptide nucleic acids (PNA) and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WIPO publications WO 00/56746 and WO 01/14398, and related publications. Methods for synthesizing oligonucleotides comprising such analogs or derivatives are disclosed, for example, in the patent publications cited above, in U.S. Pat. Nos. 5,614,622; 5,739,314; 5,955,599; 5,962,674; 6,117,992; and in WO 00/75372.

Featured herein are nucleic acids that include nucleotide sequences capable of forming a secondary structure. Examples of secondary structures are quadruplex structures, which form from subsequences rich in purines (e.g., guanines in G-quadruplex structures), and i-motif structures, which form from subsequences rich in pyrimidines (e.g., cytosines). Secondary structures can exist in different conformations, which differ in strand stoichiometry and/or strand orientation. For example, secondary structures sometimes are formed by interstrand interactions, in which the interacting strands are in the same direction (e.g., the interacting strands are oriented 5′ to 3′) or in different directions (e.g., the interacting strands are oriented 5′ to 3′ and 3′ to 5′), and sometimes are formed by intrastrand interactions. Quadruplex structures sometimes form because certain purine rich strands are capable of engaging in a slow equilibrium between a typical duplex helix structure and both unwound and non-B-form substructures. These unwound and non-B forms sometimes are referred to as “paranemic structures,” and some forms are associated with sensitivity to S1 nuclease digestion, which sometimes are referred to as “nuclease hypersensitivity elements” or “NHEs.” A quadruplex is one type of paranemic structure and certain NHEs can adopt a quadruplex structure. The entire length of the nucleic acid sometimes participates in the quadruplex structure, and a portion of the nucleic acid length (i.e., a subsequence) often forms a quadruplex structure.

The ability of guanine-rich nucleic acids of adopting G-quadruplex conformations is due to the formation of guanine tetrads through Hoogsteen hydrogen bonds. One nucleic acid sequence can give rise to different quadruplex orientations, where the different conformations depend upon conditions under which they form, such as the concentration of potassium ions present in the system and the time that the quadruplex is allowed to form. Different quadruplex conformations can be distinguished from one another using standard procedures such as chemical footprinting studies and circular dichroism signals (see e.g., U.S. application Ser. No. 10/407,449 filed Apr. 4, 2003). Also, multiple conformations can be in equilibrium with one another, and can be in equilibrium with a duplex conformation if a complementary strand exists in the system. For example, basket quadruplex conformations may be in equilibrium with intramolecular chair conformations (i.e., the latter conformation having bridging loops running orthogonal to two parallel loops and resulting from the simple folding-over of a DNA G-hairpin). The equilibrium may be shifted to favor one conformation over another such that the favored conformation is present in a higher concentration or fraction over the other conformation or other conformations. A certain conformation also may be trapped, by selectively binding the conformation over others by a compound that stabilizes the particular conformation. The terms “favor” and “trap” as used herein refer to one conformation being at a higher concentration or fraction relative to other conformations, and also refer to stabilizing the particular quadruplex conformation. The terms “hinder” or “non-trapped” as used herein refer to one conformation being at a lower concentration with respect to other conformations. One conformation may be favored or trapped over another conformation if it is present in the system at a fraction of 50% or greater, 75% or greater, or 80% or greater or 90% or greater with respect to another conformation (e.g., another quadruplex conformation, another paranemic conformation, or a duplex conformation). Conversely, one conformation may be hindered or not trapped if it is present in the system at a fraction of 50% or less, 25% or less, or 20% or less and 10% or less, with respect to another conformation.

Equilibrium can be shifted to favor one quadruplex form over another by employing a variety of methods. For example, certain bases in a quadruplex nucleic acid may be mutated to prevent the formation of one conformation. Typically, these mutations are located in tetrad regions of the quadruplex (i.e., regions in which four bases interact with one another in a planar orientation). Also, ion concentrations and the time with which a quadruplex nucleic acid is contacted with certain ions can favor one conformation over another. For example, potassium ions stabilize quadruplex structures, and higher concentrations of potassium ions and longer contact times of potassium ions with a quadruplex nucleic acid can favor one conformation over another. A particular quadruplex conformation, such as a chair conformation, can be favored with contact times of 5 minutes or less in solutions containing 100 mM potassium ions, and often 10 minutes or less, 20 minutes or less, 30 minutes or less, and 40 minutes or less. Basket conformations typically require longer contact times with potassium ions. Potassium ion concentration and the counter anion can vary, and the specific quadruplex conformations existing for a given set of conditions can be determined. Furthermore, different quadruplex structures may be distinguished, trapped and favored by probing them with molecules that favorably interact with one quadruplex form over another (e.g., TMPyP4 binds with a higher affinity to chair structures as opposed to basket structures). Quadruplex-interacting compounds sometimes bind with higher affinity to particular quadruplex structures in vitro than in vivo.

Particular nucleotide sequences in a nucleic acid often direct the type of secondary structure or structures that the nucleic acid is capable of adopting. For example, nucleic acid sequences conforming to the motif (GₐX_{b})_{c}Gₐ sometimes form an intramolecular chair G-quadruplex structure. Sometimes a is an integer between 2 and 6 and b is an integer between 1 and 4, and often, b is the integer 2 or 3. A nucleic acid often includes one or more flanking nucleotides on the 5′ and/or 3′ end of the nucleotide sequence that forms the quadruplex and are not part of the quadruplex structure. These motifs can be used to identify other quadruplex-forming sequences in regions of a genome operably linked to a gene.

Often, a nucleic acid capable of forming one or more secondary structures includes a nucleotide sequence identical to a native nucleotide sequence present in the central flap of a retrovirus. The central flap is a single-stranded region in mostly double-stranded transcription product of a retrovirus. Retroviruses include but are not limited to human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV), bovine immunodeficiency virus (BIV), murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). In an embodiment, the nucleotide sequence is from human immunodeficiency virus (HIV, e.g., HIV-1 or HIV-2) and comprises the sequences TTG₆TA (sometimes referred to as the G₆ stretch), CAG₄AA (sometimes referred to as the G₄ stretch), or both. In the latter nucleotide sequences, one or more nucleotides flanking the G₆ and G₄ stretches sometimes are substituted by another nucleotide (e.g., one or more of the TT or TA flanking the G₆ stretch or CA or AA flanking the G₄ stretch sometimes are substituted by another nucleotide). In related embodiments, the nucleic acid includes additional sequences flanking the G₆ or G₄ stretches from an HIV strain published in a publically available database (see e.g., http address hiv-web.lanl.gov), and in specific embodiments, the nucleic acid comprises or consists of the nucleotide sequence TTGGGGGGTACAGTGCAGGGGAA. The nucleic acid usually does not consist of the nucleic acid sequences TTG₆TACAGTGCA; TTG₆TACAGTGCAG₄AAA or TTG₆TACAGTGCAG₄AAAGAATAGTAGACATAATAGCAACAGAC.

A nucleic acid sometimes includes a nucleotide sequence similar to or substantially identical to a native nucleotide sequence. A similar or substantially identical nucleotide sequence may include modifications to the native sequence, such as substitutions, deletions, or insertions of one or more nucleotides. The substantially identical sequence sometimes conforms to the (GₐX_{b})_{c}Gₐ motif described above. The term “substantially identical” refers to two or more nucleic acids sharing one or more identical nucleotide sequences. Included are nucleotide sequences that sometimes are 55%, 60%, 65%, 70%, 75%, 80%, or 85% identical to a native quadruplex-forming nucleotide sequence, and often are 90% or 95% identical to the native quadruplex-forming nucleotide sequence (each identity percentage can include a 1%, 2%, 3% or 4% variance). One test for determining whether two nucleic acids are substantially identical is to determine the percentage of identical nucleotide sequences shared between the nucleic acids.

Calculations of sequence identity can be performed as follows. Sequences are aligned for optimal comparison purposes and gaps can be introduced in one or both of a first and a second nucleic acid sequence for optimal alignment. Also, non-homologous sequences can be disregarded for comparison purposes. The length of a reference sequence aligned for comparison purposes sometimes is 30% or more, 40% or more, 50% or more, often 60% or more, and more often 70%, 80%, 90%, 100% of the length of the reference sequence. The nucleotides at corresponding nucleotide positions then are compared among the two sequences. When a position in the first sequence is occupied by the same nucleotide as the corresponding position in the second sequence, the nucleotides are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two nucleotide sequences can be determined using the algorithm of Meyers & Miller, CABIOS 4:11-17 (1989), which has been incorporated into the ALIGN program (version 2.0), using a PAM120 weight residue table, a gap length penalty of 12 and a gap penalty of 4. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent conditions is hybridization in 6× sodium chloride/sodium citrate (SSC) at about 45° C., followed by one or more washes in 0.2×SSC, 0.1% SDS at 50° C. Another example of stringent conditions are hybridization in 6× sodium chloride/sodium citrate (SSC) at about 45° C., followed by one or more washes in 0.2×SSC, 0.1% SDS at 55° C. A further example of stringent conditions is hybridization in 6× sodium chloride/sodium citrate (SSC) at about 45° C., followed by one or more washes in 0.2×SSC, 0.1% SDS at 60° C. Often, stringent conditions are hybridization in 6× sodium chloride/sodium citrate (SSC) at about 45° C., followed by one or more washes in 0.2×SSC, 0.1% SDS at 65° C. Also, stringency conditions include hybridization in 0.5M sodium phosphate, 7% SDS at 65° C., followed by one or more washes at 0.2×SSC, 1% SDS at 65° C.

Also, nucleotide sequences of native quadruplex-forming nucleotide sequences may be used as “query sequences” to perform a search against public databases to identify related sequences. Such searches can be performed using the NBLAST and XBLAST programs (version 2.0) of Altschul, et al., J. Mol. Biol. 215:403410 (1990). BLAST nucleotide searches can be performed with the NBLAST program, score=100, wordlength=12 to obtain homologous nucleotide sequences. To obtain gapped alignments for comparison purposes, Gapped BLAST can be utilized as described in Altschul, et al., Nucleic Acids Res. 25(17):3389-3402 (1997). When utilizing BLAST and Gapped BLAST programs, default parameters of the respective programs (e.g., XBLAST and NBLAST) can be used (see, http address www.ncbi.nlm.nih.gov).

Test Molecules and Candidate Molecules

The nucleic acid is contacted with one or more test molecules to identify candidate molecules. Molecules often are organic or inorganic compounds having a molecular weight of 10,000 grams per mole or less, and sometimes having a molecular weight of 5,000 grams per mole or less, 1,000 grams per mole or less, or 500 grams per mole or less. Also included are salts, esters, and other pharmaceutically acceptable forms of the compounds. Compounds that interact with nucleic acids are known in the art (see, e.g., Hurley, Nature Rev. Cancer 2:188-200 (2002); Anantha, et al., Biochemistry Vol. 37, No. 9:2709-2714 (1998); and Ren, et al., Biochemistry 38:16067-16075 (1999)).

Compounds can be obtained using known combinatorial library methods, including spatially addressable parallel solid phase or solution phase libraries; synthetic library methods requiring deconvolution; “one-bead one-compound” library methods; and synthetic library methods using affinity chromatography selection. Examples of methods for synthesizing molecular libraries are described, for example, in DeWitt, et al., Proc. Natl. Acad. Sci. U.S.A. 90:6909 (1993); Erb, et al., Proc. Natl. Acad. Sci. USA 91:11422 (1994); Zuckermann, et al., J. Med. Chem. 37:2678 (1994); Cho, et al., Science 261:1303 (1993); Carrell, et al., Angew. Chem. Int. Ed. Engl. 33:2059 (1994); Carell, et al., Angew. Chem. Int. Ed. Engl. 33:2061 (1994); and Gallop, et al., J. Med. Chem. 37:1233 (1994).

In addition to an organic and inorganic compound, a molecule sometimes is a nucleic acid, a catalytic nucleic acid (e.g., a ribozyme), a small interfering RNA (siRNA), a nucleotide, a nucleotide analog, a polypeptide, an antibody, or a peptide mimetic. Methods for making and using these molecules are known in the art. For example, methods for making ribozymes and assessing ribozyme activity are described (see e.g., U.S. Pat. Nos. 5,093,246; 4,987,071; and 5,116,742; Haselhoff & Gerlach, Nature 334:585-591 (1988) and Bartel & Szostak, Science 261:1411-1418 (1993)). Also, methods for generating siRNA are known (see e.g., Elbashir, et al,. Methods 26:199-213 (2002) and http address www.dharmacon.com) and peptide mimetic libraries are described (see, e.g., Zuckermann, et al., J. Med. Chem. 37:2678-2685 (1994)).

Test molecules sometimes are capable of end-stacking with or intercalating between one or more G-tetrads of a G-quadruplex, such as a moiety comprising a planar or polycyclic structure, for example. Examples of such moieties are anthraquinone, acridone, napthyl, pheoxazine, xanthone, benzoxazole, phenathiazine, phenazine, benzothiazole, acridine, dibenzofuran, benzimidazole, fluorenone, fluorene, and phenanthroline. In another embodiment, the test molecule includes a moiety that is a duplex DNA intercalator, capable of binding to a duplex DNA region adjacent to a secondary structure in the nucleic acid, such as a moiety having a planar or polycyclic structure (e.g., an intercalator listed previously). In a related embodiment, the moiety is capable of groove-binding to a duplex region in the nucleic acid, such as a polypeptide or sugar-based moiety capable of groove binding. In other embodiments, a moiety is capable of binding to an amino acid of a nucleic acid binding protein, such as a nucleotide or a nucleotide mimetic, or a carbonyl-, acetal-, or imine-containing moiety.

A molecule sometimes interacts with two or more target regions in a nucleic acid. Such molecules often comprise two or more moieties that independently interact with target regions and are joined by a linker. A linker joining the moieties often is 7.5 Å to 40 Å in length, often comprises between 5 and 20 atoms, often is flexible, and sometimes is constrained (e.g., in a conformation that follows the groove of duplex DNA). The linker sometimes comprises polyamide or polysaccharide (e.g., comprising amino saccharide units) moieties, and typically includes known linkage functionalities such as those independently selected from amide, ester, ether, amine, sulfide, sulfonamide, alkyl or aryl, for example. The molecule sometimes is bifunctional or multifunctional, with each functional moiety targets the same type of nucleic acid structure and each functional moiety is separated by a linker. The latter embodiment is useful for “gluing” together central flap regions of retroviruses and thereby inhibiting viral proliferation.

An interaction between the test molecule and the nucleic acid sometimes is covalent and often is non-covalent (e.g., hydrogen bond, hydrophobic interaction, Van der Waals interaction) and at times is processing of the nucleic acid (e.g., covalent attachment of nucleic acids to one another). The interaction sometimes is detected by processes that monitor quadruplex stabilization, such as CD assays, chemical footprinting assays, and polymerase arrest assays described hereafter.

Featured herein is structural information descriptive of the candidate molecules and therapeutics identified by the processes described herein. In certain embodiments, information descriptive of candidate molecule structure (e.g., chemical formula or sequence information) sometimes is stored and/or renditioned as an image or as three-dimensional coordinates. The information often is stored and/or renditioned in computer readable form and sometimes is stored and organized in a database. In certain embodiments, the information may be transferred from one location to another using a physical medium (e.g., paper) or a computer readable medium (e.g., optical and/or magnetic storage or transmission medium, floppy disk, hard disk, random access memory, computer processing unit, facsimile signal, satellite signal, transmission over an internet or transmission over the world-wide web).

Screening Processes

Candidate molecules are contacted with the nucleic acid in the assay system, where the term “contacting” refers to placing a candidate molecule in close proximity to a nucleic acid and allowing the assay components to collide with one another, often by diffusion. Contacting these assay components with one another can be accomplished by adding them to a body of fluid or in a reaction vessel, for example. The components in the system may be mixed in variety of manners, such as by oscillating a vessel, subjecting a vessel to a vortex generating apparatus, repeated mixing with a pipette or pipettes, or by passing fluid containing one assay component over a surface having another assay component immobilized thereon, for example.

As used herein, the term “system” refers to an environment that receives the assay components, which includes, for example, microtitre plates (e.g., 96-well or 384-well plates), silicon chips having molecules immobilized thereon and optionally oriented in an array (see, e.g., U.S. Pat. No. 6,261,776 and Fodor, Nature 364:555-556 (1993)), and microfluidic devices (see, e.g., U.S. Pat. Nos. 6,440,722; 6,429,025; 6,379,974; and 6,316,781). The system can include attendant equipment for carrying out the assays, such as signal detectors, robotic platforms, and pipette dispensers. Sometimes the system includes one or more cells, and sometimes the system is a subject.

One or more assay components (e.g., the nucleic acid, candidate molecule or nucleic acid binding protein) sometimes are immobilized to a solid support. The attachment between an assay component and the solid support often is covalent and sometimes is non-covalent (see, e.g., U.S. Pat. No. 6,022,688 for non-covalent attachments). The solid support often is one or more surfaces of the system, such as one or more surfaces in each well of a microtiter plate, a surface of a silicon wafer, a surface of a bead (see, e.g., Lam, Nature 354: 82-84 (1991)) optionally linked to another solid support, or a channel in a microfluidic device, for example. Types of solid supports, linker molecules for covalent and non-covalent attachments to solid supports, and methods for immobilizing nucleic acids and other molecules to solid supports are known (see, e.g., U.S. Pat. Nos. 6,261,776; 5,900,481; 6,133,436; and 6,022,688; and WIPO publication WO 01/18234).

Protein molecules sometime are contacted with the nucleic acid. Polypeptide molecules sometimes are added to the system in free form, and sometimes are linked to a solid support or another molecule. For example, polypeptide test molecules sometimes are linked to a phage via a phage coat protein. The latter embodiment often is accomplished by using a phage display system, where nucleic acids linked to a solid support are contacted with phages that display different polypeptide candidate molecules. Phages displaying polypeptide candidate molecules that interact with the immobilized nucleic acids adhere to the solid support, and phage nucleic acids corresponding to the adhered phages then are isolated and sequenced to determine the sequence of the polypeptide test molecules that interacted with the immobilized nucleic acids. Methods for displaying a wide variety of peptides or proteins as fusions with bacteriophage coat proteins are known (Scott and Smith, Science 249:386-390 (1990); Devlin, Science 249:404-406 (1990); Cwirla, et al., Proc. Natl. Acad. Sci. 87:6378-6382 (1990); Felici, J. Mol. Biol. 222:301-310 (1991); U.S. Pat. Nos. 5,096,815 and 5,198,346; U.S. Pat. Nos. 5,223,409; 5,403,484; 5,571,698; and 5,766,905). Methods also are available for linking the test polypeptide to the N-terminus or the C-terminus of the phage [symbol] protein.

A signal generated by the system when a candidate molecule binds to a nucleic acid and/or a nucleic acid binding protein often scales directly with a range of increasing nucleic acid, nucleic acid binding protein, or candidate molecule concentrations. Signal intensity often exhibits a hyperbolic relationship when plotted as a function of nucleic acid, candidate molecule, or nucleic acid binding protein concentrations. The signal sometimes is increased relative to background signal levels when a candidate molecule binds to a nucleic acid and/or a nucleic acid binding protein, and sometimes the signal decreases relative to background signal levels under such circumstances. The candidate molecules often interact with the nucleic acid and/or nucleic acid binding protein by reversible binding, and sometimes interact with irreversible binding. For example, the candidate molecule may reversibly form a covalent bond between a portion of the candidate molecule and an amino acid side chain in the protein (e.g., a lysine), depending on the chemical structure of the candidate molecule.

Candidate molecules often are identified as interacting with the nucleic acid and/or nucleic acid binding protein when the signal produced in a system containing the candidate molecule is different than the signal produced in a system not containing the candidate molecule. While background signals may be assessed each time a new candidate molecule, nucleic acid, or nucleic acid binding protein is probed by the assay, detecting the background signal is not required each time a new test molecule or test nucleic acid is assayed. Control assays also can be performed to determine background signals and to rule out false positive results and false negative results. Such control assays often do not include one or more assay components included in other assays (e.g., a control assay sample sometimes does not include a candidate molecule, a nucleic acid, or a protein that interacts with the nucleic acid).

In addition to determining whether a candidate molecule gives rise to a different signal, the affinity of the interaction between the candidate molecule with the nucleic acid and/or nucleic acid binding protein sometimes is quantified. IC₅₀, K_{d}, or Kᵢ threshold values sometimes are compared to the measured IC₅₀ or K_{d} values for each interaction, and thereby are used to identify a candidate molecule that interacts with the nucleic acid or nucleic acid binding protein and modulates the biological activity. For example, IC₅₀ or K_{d} threshold values of 10 μM or less, 1 μM or less, and 100 nM or less often are utilized, and sometimes threshold values of 10 nM or less, 1 nM or less, 100 pM or less, and 10 pM or less are utilized to identify candidate molecules that interact with nucleic acids and/or binding proteins and modulate the biological activity.

Candidate molecules identified by the competition assays described herein sometimes are pre-screened or post-screened in other in vitro or in vivo assays. Candidate molecules and nucleic acids can be added to an assay system in any order to determine whether the candidate molecule modulates the biological activity of the nucleic acid. For example, a candidate molecule sometimes is added to an assay system before, simultaneously, or after a nucleic acid is added.

For example, fluorescence assays, gel mobility shift assays (see, e.g., Jin & Pike, Mol. Endocrinol. 10:196-205 (1996) and Postel, J. Biol. Chem. 274:22821-22829 (1999)), polymerase arrest assays, transcription reporter assays, DNA cleavage assays, protein binding and apoptosis assays (see, e.g., Amersham Biosciences (Piscataway, N.J.)) sometimes are utilized. Also, topoisomerase assays sometimes are utilized subsequently to determine whether the quadruplex interacting molecules have a topoisomerase pathway activity (see, e.g., TopoGEN, Inc. (Columbus, Ohio)).

A fluorescence interaction assay is useful for identifying candidate molecules that interact with DNA capable of forming a quadruplex structure. In particular, such assays are useful in in vitro high-throughput assays and in gel electrophoretic mobility shift assays. Such methods sometimes comprise contacting a sample comprising a nucleic acid with a test molecule, where the nucleic acid includes or consists of nucleotide sequence that is identical or substantially similar to a native nucleotide sequence capable of forming a G-quadruplex structure. One or more nucleoside moieties in the native nucleotide sequence sometimes are substituted with a fluorescent nucleoside analog. Examples of such fluorescent nucleoside analogs are 2-amino purine (e.g., 2-amino adenosine), pyrrolo-C, 6-MAP, and furano-dT (for other examples, see http address www.glenresearch.com/GlenReports/GR15-13.html). A fluorescent signal generated by the sample is detected after the sample is contacted by the test molecule, and the test molecule is identified as a candidate molecule that interacts with the nucleic acid when the fluorescent signal detected before the sample is contacted with the test molecule differs from the fluorescent signal detected after the sample is contacted with the test molecule. Fluctuations sometimes are reduced fluorescence intensity at a particular wavelength, and sometimes are shifts in the wavelengths at which fluorescence is detected. Often, the labeled strand is hybridized with a complementary strand and any fluctuations in fluorescence are detected upon hybridization, and the labeled hybrid then is contacted with test molecules and fluctuations in fluorescence are detected to determine which of the test molecules interact with the labeled nucleic acid. In certain embodiments, the sample is contacted with a nucleic acid binding protein such as NM23-H2, Sp1, CNBP and/or hnRNP_{κ} before, at the same time, or after the sample is contacted with the test molecule. In other embodiments, the labeled nucleic acid is interacted with test molecules or proteins and the reaction products then are subjected to a gel electrophoretic mobility shift assay.

Another example of a fluorescence interaction assay is a system that includes a nucleic acid, a signal molecule, and a candidate or test molecule. The signal molecule generates a fluorescent signal when bound to the nucleic acid (e.g., N-methylmesoporphyrin IX (NMM)), and the signal is altered when a candidate compound competes with the signal molecule for binding to the nucleic acid. An alteration in the signal when a candidate molecule is present as compared to when the candidate molecule is not present identifies the candidate molecule as a nucleic acid-interacting molecule. 50 μl of nucleic acid is added in 96-well plate. A candidate molecule also is added in varying concentrations. A typical assay is carried out in 100 μl of 20 mM HEPES buffer, pH 7.0, 140 mM NaCl, and 100 mM KCl. 50 μl of the signal molecule NMM then is added for a final concentration of 3 μM. NMM is obtained from Frontier Scientific Inc, Logan, Utah. Fluorescence is measured at an excitation wavelength of 420 nm and an emission wavelength of 660 nm using a FluroStar 2000 fluorometer (BMG Labtechnologies, Durham, N.C.). Fluorescence often is plotted as a function of concentration of the candidate molecule or nucleic acid and maximum fluorescent signals for NMM are assessed in the absence of these molecules.

DNA cleavage assays are useful for determining at which sites of a nucleic acid a nucleic acid binding protein interacts, for example. DNA cleavage assays have been reported (e.g., Postel, J. Biol. Chem., 274:22821-22829 (1999)). In general, a detectable label is incorporated at one portion of the nucleic acid and the label is separated from another portion of the nucleic acid having no detectable label or a different detectable label upon cleavage. Examples of detectable labels are known, such as fluorophores (e.g., Anantha, et al., Biochemistry 37:2709-2714 (1998) and Qu & Chaires, Methods Enzymol. 321:353-369 (2000)), fluorescent nucleotide analogs described above, NMR spectral shifts (see, e.g., Arthanari & Bolton, Anti-Cancer Drug Design 14:317-326 (1999)), fluorescence resonance energy transfers (see, e.g., Simonsson & Sjöback, J. Biol. Chem. 274:17379-17383 (1999)), a radioactive isotope (e.g., ¹²⁵I, ¹³¹I, ³⁵S, ³²P, ¹⁴C or ³H); a light scattering label (see, e.g., U.S. Pat. No. 6,214,560; Genicon Sciences Corporation, San Diego, Calif.); an enzymic or protein label (e.g., green fluorescent protein (GFP) or peroxidase), or another chromogenic label or dye. The nucleic acid also can be linked to two fluorophores for a fluorescence resonance energy transfer (FRET) assay, where one fluorophore emits light at a wavelength at which the other fluorophore is excited, where such fluorescence energy transfer occurs when the nucleic acid is intact and does not occur when the nucleic acid is cleaved by a nucleic acid binding protein. Similarly, a candidate molecule linked to a nucleic acid binding protein can be detected by detecting the candidate molecule bound to the protein or a detectable label bound to a candidate molecule linked to a binding protein.

A gel electrophoretic mobility shift assay (EMSA) is useful for determining whether a nucleic acid forms a quadruplex and whether a nucleotide sequence is quadruplex-destabilizing. EMSA is conducted as described previously (Jin & Pike, Mol. Endocrinol. 10: 196-205 (1996)) with minor modifications. Synthetic single-stranded oligonucleotides are labeled in the 5′ terminus with T4-kinase in the presence of [γ-³²P] ATP (1,000 mCi/mmol, Amersham Life Science) and purified through a sephadex column. ³²P-labeled oligonucleotides (˜30,000 cpm) then are incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25×Tris borate-EDTA buffer (0.25×TBE, 1×TBE is 89 mM Tris-borate, pH 8.0, 1 mM EDTA). The gel is dried and each band is quantified using a phosphorimager.

Another example of an EMSA assay is performed as follows. Ten microliter reactions are assembled in Reaction Buffer (50 mM Tris-HCl, pH 7.9, 0.5 mM dithiothreitol, and 50 mg/ml bovine serum albumin). MgCl₂, KCl, EDTA, protease K, and ATP are added. Radiolabeled DNA or fluorescently labeled DNA (described above) and NM23-H2 in storage buffer (20 mM Hepes, pH 7.9, 5 mM MgCl₂, 0.1 mM EDTA, 0.1 M KCl, 1 mM dithiothreitol, 20% glycerol, and protease inhibitors (Postel, et al., Mol. Cell. Biol. 9:5123-5133 (1989)) are added last, and the reactions are incubated for 15 minutes at room temperature. To separate the protein-DNA complexes, the reactions are loaded onto 5% native polyacrylamide gels and electrophoresed in 0.53 TBE buffer (45 mM Tris borate, pH 8.3, 1.25 mM EDTA) at room temperature for 30 minutes at 100 V. Gels are vacuum-dried and exposed onto XAR (Eastman Kodak Co.) film.

Chemical footprinting assays are useful for assessing quadruplex structure. Quadruplex structure is assessed by determining which nucleotides in a nucleic acid is protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoathanol:water:NaOAc (3 M); 1:6:7; v:v:v). Following ethanol precipitation (twice) and piperidine cleavage, the reactions are separated on a preparative gel (16%) and visualized on a phosphorimager.

A polymerase arrest assay is useful for determining whether transcription is modulated by a candidate molecule and/or a nucleic acid binding protein. Such an assay includes a template nucleic acid, which often comprises a quadruplex forming sequence, and a primer nucleic acid which hybridizes to the template nucleic acid 5′ of the quadruplex-forming sequence. The primer is extended by a polymerase (e.g., Taq polymerase), which advances from the primer along the template nucleic acid. In this assay, a quadruplex structure can block or arrest the advance of the enzyme, leading to shorter transcription fragments. Also, the arrest assay may be conducted at a variety of temperatures, including 45° C. and 60° C., and at a variety of ion concentrations. An example of the Taq polymerase stop assay is described in Han, et al., Nucl. Acids Res. 27:537-542 (1999), which is a modification of that used by Weitzmann, et al., J. Biol. Chem. 271, 20958-20964 (1996). Briefly, a reaction mixture of template DNA (50 nM), Tris.HCl (50 mM), MgCl₂ (10 mM), DTT (0.5 mM), EDTA (0.1 mM), BSA (60 ng), and 5′-end-labeled quadruplex nucleic acid (˜18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl), and 10 mg bromophenol blue), the reactions are separated on a preparative gel (12%) and visualized on a phosphorimager. Adenine sequencing (indicated by “A” at the top of the gel) is performed using double-stranded DNA Cycle Sequencing System from Life Technologies. The general sequence for the template strands is TCCAACTATGTATAC-INSERT-TTAGCGACACGCAATTGCTATAGTGAGTCGTATTA. Bands on the gel that exhibit slower mobility are indicative of quadruplex formation.

Another example of a polymerase arrest assay can be used in a medium to high throughput format. In this assay embodiment, a 5′-fluorescent-labeled (FAM) primer (P45, 15 nM) is mixed with template DNA (15 nM) in a Tris-HCL buffer (15 mM Tris, pH 7.5) containing 10 mM MgCl₂, 0.1 mM EDTA and 0.1 mM mixed deoxynucleotide triphosphates (dNTP's). The assay is performed by copying a template with a polymerase, where the copy is primed from a fluoresently labeled primer nucleic acid (e.g., a suitable fluoescent label is FAM). The template comprises a sequence from the central flap region capable of forming a quadruplex and the primer is a smaller nucleic acid complementary to a region upstream of the sequence in the template capable of forming the quadruplex. In an example of a template and primer for detecting quadruplex formation in the CMYC promoter, the FAM-P45 primer (5′-6FAM-AGTCTGACTGACTGTACGTAGCTAATACGACTCACTATAGCAATT-3′) and the template DNA (5′-TCCAACTATGTATACTGGGGA GGGTGGGGAGGGTGGGGAAGGTT AGCGACACGCAATTGCTATAG TGAGTCGTATTAGCTACGTACAGTCAGTCAGACT-3′) are synthesized and HPLC purified by Applied Biosystems. The mixture is denatured at 95° C. for 5 minutes and, after cooling down to room temperature, is incubated at 37° C. for 15 minutes. After cooling down to room temperature, 1 mM KCl₂ and the test compound (various concentrations) are added and the mixture incubated for 15 minutes at room temperature. The primer extension is performed by adding 10 mM KCl and Taq DNA Polymerase (2.5 U/reaction, Promega) and incubating at 70° C. for 30 minutes. The reaction is stopped by adding 1 μl of the reaction mixture to 10 μl Hi-Di Formamide mixed and 0.25 μl LIZ120 size standard. Hi-Di Formamide and LIZ120 size standard are purchased from Applied Biosystems. The partially extended quadruplex arrest product is between 61 or 62 bases long and the full-length extended product is 99 bases long. The products are separated and analyzed using capillary electrophoresis. Capillary electrophoresis is performed using an ABI PRISM 3100-Avant Genetic Analyzer.

Certain arrest assays are performed in cells. In a transcription reporter assay, test quadruplex DNA is coupled to a reporter system, such that a formation or stabilization of a quadruplex structure can modulate a reporter signal. An example of such a system is a reporter expression system in which a polypeptide, such as luciferase or green fluorescent protein (GFP), is expressed by a gene operably linked to the potential quadruplex forming nucleic acid and expression of the polypeptide can be detected. As used herein, the term “operably linked” refers to a nucleotide sequence which is regulated by a sequence comprising the potential quadruplex forming nucleic acid. A sequence may be operably linked when it is on the same nucleic acid as the quadruplex DNA, or on a different nucleic acid. An exemplary luciferase reporter system is described herein. A luciferase promoter assay described in He, et al., Science 281:1509-1512 (1998) often is utilized for the study of quadruplex formation. Specifically, a vector utilized for the assay is set forth in reference 11 of the He, et al., document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

Circular dichroism (CD) sometimes is utilized to determine whether another molecule interacts with a quadruplex nucleic acid. CD is particularly useful for determining whether a candidate molecule interacts with a nucleic acid in vitro. In certain embodiments, a candidate molecule is added to a DNA sample (5 μM each) in a buffer containing 10 mM potassium phosphate (pH 7.2) and 10 or 250 mM KCl at 37° C. and then allowed to stand for 5 min at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for DNA alone, candidate molecule alone, and the DNA with the candidate molecule are generated to determine the presence or absence of an interaction (see e.g. Datta et al., JACS 123:9612-9619 (2001)). Spectra are arranged to represent the average of eight scans recorded at 100 nm/min. In certain embodiments, CD signals are monitored as a function of temperature. For example, CD signals indicative of the presence of a quadruplex structure in a sample can be determined and the loss or shift of those signals can be followed as a function of increasing temperature to determine at which temperature the quadruplex structure melts. The signals often are monitored in the presence of test molecule and in the absence of test molecule, and stabilizing molecules that increase the melting temperature of the quadruplex structure are identified as candidate molecules.

Inhibitory activity of the candidate molecules on viral proliferation sometimes is assessed. Viral titres are monitored by known processes (see e.g. Kim et al., J Virol 72: 811-816 (1998) and Soneoka et al., Nucleic Acids Res. 23:628-33 (1995)). Briefly, cells (e.g., 293T cells) are seeded on 6 cm dishes and 24 hours later they are transiently transfected by overnight calcium phosphate treatment. The medium sometimes is replaced 12 hours post-transfection and supernatants often are harvested 48 hours post-transfection, filtered (through 0.22 or 0.45 μm filters) and titered by transduction of 293T cells. Supernatant at appropriate dilutions of the original stock often is added to the cells (plated onto 6 or 12 well plates 24 hours prior to transduction). 8 μg/ml Polybrene (Sigma) often is added to each well and 48 hours post transduction viral titres are determined by X-gal staining.

Utilization of Candidate Molecules as Therapeutics

Because quadruplexes are regulators of biological processes such as oncogene transcription, modulators of quadruplex biological activity can be utilized as cancer therapeutics. For example, molecules that stabilize quadruplex structures can exert a therapeutic effect for certain cell proliferative disorders and related conditions because quadruplex structures typically down-regulate the oncogene expression which can cause cell proliferative disorders. Quadruplex-interacting candidate molecules can exert a biological effect according to different mechanisms, which include, for example, stabilizing a native quadruplex structure, inhibiting conversion of a native quadruplex to duplex DNA, and stabilizing a native quadruplex structure having a quadruplex-destabilizing nucleotide substitution. Thus, quadruplex interacting candidate molecules described herein may be administered to cells, tissues, or organisms, thereby down-regulating oncogene transcription and treating cell proliferative disorders. The terms “treating,” “treatment” and “therapeutic effect” as used herein refer to reducing or stopping a viral proliferation rate (e.g., slowing or halting viral titre) and sometimes refers to alleviating, completely or in part, a viral proliferation condition.

Thus, featured are methods for reducing viral proliferation or for treating or alleviating a viral disorder, which comprise contacting a system infected with a retrovirus with a candidate molecule identified by the processes described herein. The system may be infected with any of the retroviruses described above. The system sometimes is a group of cells or one or more tissues, and often is a subject in need of an antiviral treatment. A subject often is a mammal such as a mouse, rat, monkey, or human. In an embodiment, provided is a method for reducing and/or treating HIV infection by administering a candidate molecule identified herein to a subject in need thereof, thereby reducing the HIV titres in the system and alleviating infection.

Any suitable formulation of the candidate molecules described herein can be prepared for administration. Any suitable route of administration may be used, including but not limited to oral, parenteral, intravenous, intramuscular, topical and subcutaneous routes.

In cases where candidate molecules are sufficiently basic or acidic to form stable nontoxic acid or base salts, administration of the candidate molecules as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example by reacting a sufficiently basic candidate molecule such as an amine with a suitable acid affording a physiologically acceptable anion. Alkali metal (e.g., sodium, potassium or lithium) or alkaline earth metal (e.g., calcium) salts of carboxylic acids also are made.

In one embodiment, a candidate molecule is administered systemically (e.g., orally) in combination with a pharmaceutically acceptable vehicle such as an inert diluent or an assimilable edible carrier. They may be enclosed in hard or soft shell gelatin capsules, compressed into tablets, or incorporated directly with the food of the patient's diet. For oral therapeutic administration, the active candidate molecule may be combined with one or more excipients and used in the form of ingestible tablets, buccal tablets, troches, capsules, elixirs, suspensions, syrups, wafers, and the like. Such compositions and preparations should contain at least 0.1% of active candidate molecule. The percentage of the compositions and preparations may be varied and may conveniently be between about 2 to about 60% of the weight of a given unit dosage form. The amount of active candidate molecule in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active candidate molecule, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active candidate molecule may be incorporated into sustained-release preparations and devices.

The active candidate molecule also may be administered intravenously or intraperitoneally by infusion or injection. Solutions of the active candidate molecule or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The candidate molecule is sometimes prepared as a polymatrix-containing formulation for such administration (e.g., a liposome or microsome). Liposomes are described for example in U.S. Pat. No. 5,703,055 (Feigner, et al.) and Gregoriadis, Liposome Technology vols. I to III (2nd ed. 1993).

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active candidate molecule in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present candidate molecules may be applied in liquid form. Candidate molecules often are administered as compositions or formulations, in combination with a dermatologically acceptable carrier, which may be a solid or a liquid. Examples of useful dermatological compositions used to deliver candidate molecules to the skin are known (see, e.g., Jacquet, et al. (U.S. Pat. No. 4,608,392), Geria (U.S. Pat. No. 4,992,478), Smith, et al (U.S. Pat. No. 4,559,157) and Wortzman (U.S. Pat. No. 4,820,508).

Candidate molecules may be formulated with a solid carrier, which include finely divided solids such as talc, clay, microcrystalline cellulose, silica, alumina and the like. Useful liquid carriers include water, alcohols or glycols or water-alcohol/glycol blends, in which the present candidate molecules can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Generally, the concentration of the candidate molecule in a liquid composition often is from about 0.1 wt % to about 25 wt %, sometimes from about 0.5 wt % to about 10 wt %. The concentration in a semi-solid or solid composition such as a gel or a powder often is about 0.1 wt % to about 5 wt %, sometimes about 0.5 wt % to about 2.5 wt %. A candidate molecule composition may be prepared as a unit dosage form, which is prepared according to conventional techniques known in the pharmaceutical industry. In general terms, such techniques include bringing a candidate molecule into association with pharmaceutical carrier(s) and/or excipient(s) in liquid form or finely divided solid form, or both, and then shaping the product if required. The candidate molecule composition may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

The amount of the candidate molecule, or an active salt or derivative thereof, required for use in treatment will vary not only with the particular salt selected but also with the route of administration, the nature of the condition being treated and the age and condition of the patient and will be ultimately at the discretion of the attendant physician or clinician.

A useful candidate molecule dosage often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD₅₀ (the dose lethal to 50% of the population) and the ED₅₀ (the dose therapeutically effective in 50% of the population) of a candidate molecule. The dose ratio between a toxic and therapeutic effect is the therapeutic index and it can be expressed as the ratio ED₅₀/LD₅₀. The candidate molecule dosage often lies within a range of circulating concentrations for which the ED₅₀ is associated with little or no toxicity. The dosage may vary within this range depending upon the dosage form employed and the route of administration utilized. For any candidate molecules used in the methods described herein, the therapeutically effective dose can be estimated initially from cell culture assays. A dose sometimes is formulated to achieve a circulating plasma concentration range covering the IC₅₀ (i.e., the concentration of the test candidate molecule which achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” candidate molecule in the serum of the test subject. Such assays may utilize antibody mimics and/or “biosensors” generated by molecular imprinting techniques. The candidate molecule is used as a template, or “imprinting molecule”, to spatially organize polymerizable monomers prior to their polymerization with catalytic reagents. Subsequent removal of the imprinted molecule leaves a polymer matrix which contains a repeated “negative image” of the candidate molecule and is able to selectively [symbol] the molecule under biological assay conditions (see, e.g., Ansell, et al., Current Opinion in Biotechnology 7: 89-94 (1996) and in Shea, Trends in Polymer Science 2: 166-173 (1994)). Such “imprinted” affinity matrixes are amenable to ligand-binding assays, whereby the immobilized monoclonal antibody component is replaced by an appropriately imprinted matrix [symbol] e.g., Viatakis, et al., Nature 361: 645-647 (1993)). Through the use of isotope-labeling, “free” concentration of candidate molecule can be readily monitored and used in calculations of IC₅₀. Such “imprinted” affinity matrixes can also be designed to include fluorescent groups whose photon-emitting properties measurably change upon local and selective binding of candidate molecule. These changes can be readily assayed in real time using appropriate fiber optic devices, in turn allowing the dose in a test subject to be quickly optimized based on its individual IC₅₀. An example of such a “biosensor” is discussed in Kriz, et al., Analytical Chemistry 67: 2142-2144 (1995).

Exemplary doses include milligram or microgram amounts of the candidate molecule per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific candidate molecule employed, the age, body weight, general health, gender, and diet of the subject, the time of administration, the route of administration, the rate of excretion, any drug combination, and the degree of expression or activity to be modulated.

Each document and publication cited is incorporated herein by reference in its entirety, including all figures, drawings, tables, text, and documents and publications referenced therein. The priority document, U.S. application No. 60/419,456 filed Oct. 18, 2002, also is incorporated herein by reference.

1. A method for identifying an antiviral candidate molecule, which comprises contacting a test molecule with a nucleic acid comprising a nucleotide sequence identical to or substantially identical to a nucleotide sequence in a central flap nucleic acid sequence of a retrovirus, wherein the nucleic acid comprises a quadruplex structure, and
detecting an interaction between the test molecule and the nucleic acid, whereby a test molecule that interacts with the nucleic acid is identified as an antiviral candidate molecule.

2. The method of claim 1, wherein the retrovirus is selected from the group consisting of human immunodeficiency virus (HIV), simian immunodeficiency virus (SUV), visna/maedi virus (VMV), caprine arthritis-encephalitis virus (CAEV), equine infectious anaemia virus (EIAV), feline immunodeficiency virus (FIV), bovine immunodeficiency virus (BIV), murine leukemia virus (MLV), human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), avian myelocytomatosis virus-29 (MC29), and avian erythroblastosis virus (AEV).

3. The method of claim 1, wherein the retrovirus is HIV.

4. The method of claim 1, wherein the nucleic acid comprises the nucleotide sequence TTG₆TA (SEQ ID NO:1).

5. The method of claim 1, wherein the nucleic acid comprises the nucleotide sequence CAG₄AA (SEQ ID NO:2).

6. The method of claim 1, wherein the nucleic acid comprises the nucleotide sequence TTG₆TACAGTGCAG₄AA (SEQ ID NO:3).

7. The method of claim 1, wherein the nucleic acid is incubated in a solution comprising potassium ions.

8. The method of claim 1, wherein the quadruplex is an intermolecular structure.

9. The method of claim 1, wherein the quadruplex is an intermolecular parallel structure.

10. The method of claim 1, wherein the quadruplex is an intermolecular structure formed by a dimer of two intramolecular hairpin structures.

11. The method of claim 1, wherein the interaction is detected by circular dichroism.

12. The method of claim 1, wherein the interaction is binding of the test molecule to the nucleic acid.

13. Information characterizing the structure of an antiviral candidate molecule identified by the method of claim 1.

14. A method for inhibiting retroviral proliferation in a system, which comprises contacting a system comprising a retrovirus with an antiviral candidate molecule identified by the method of claim 1; whereby the candidate molecule inhibits retroviral proliferation in the system.

15. The method of claim 14, wherein the system is a cell.

16. The method of claim 14, wherein the system is a subject.


SEQUENCE LISTING (10 sequences)

SEQ ID NO: 1 - DNA, 10 bp, Human immunodeficiency virus
ttggggggta

SEQ ID NO: 2 - DNA, 8 bp, Human immunodeficiency virus
caggggaa

SEQ ID NO: 3 - DNA, 23 bp, Human immunodeficiency virus
ttggggggtacagtgcaggggaa

SEQ ID NO: 4 - DNA, 17 bp, Human immunodeficiency virus
ttggggggtacagtgca

SEQ ID NO: 5 - DNA, 24 bp, Human immunodeficiency virus
ttggggggtacagtgcaggggaaa

SEQ ID NO: 6 - DNA, 50 bp, Human immunodeficiency virus
ttggggggtacagtgcaggggaaagaatagtagacataatagcaacagac

SEQ ID NO: 7 - DNA, 15 bp, Artificial Sequence
  Primer
tccaactatgtatac

SEQ ID NO: 8 - DNA, 35 bp, Artificial Sequence
  Primer
ttagcgacacgcaattgctatagtgagtcgtatta

SEQ ID NO: 9 - DNA, 45 bp, Artificial Sequence
  Primer
agtctgactgactgtacgtagctaatacgactcactatagcaatt

SEQ ID NO: 10 - DNA, 99 bp, Artificial Sequence
  Primer
tccaactatgtatactggggagggtggggagggtggggaaggttagcgacacgcaattgctatagtgagtcgtattagctacgtacagtcagtcagact